(19) United States
(12) Patent Application Publication
(10) Pub. No.: US 2025/0136906 A1
(43) Pub. Date: May 1, 2025

(54) METHOD AND DEVICE SUITABLE FOR GROWING ALGAE

(71) Applicant: ARIEL SCIENTIFIC INNOVATIONS, LTD., Ariel (IL)

(72) Inventors: Yaakov ANKER, Salit (IL); Alexander GIMBURG, Itamar (IL); Erez Katz, Salit (IL); Sharon SASON, Ramat Gan (IL)

(21) Appl. No.: 18/835,808

(22) PCT Filed: Feb. 22, 2023
(86) PCT No.: PCT/IB2023/051609

§ 371 Date: Aug. 5, 2024

Related U.S. Application Data
(60) Provisional application No. 63/312,935, filed on Feb. 23, 2022.

(51) Int. Cl. C12M 1/00; C12M 1/12

(52) U.S. Cl. CPC C12M 21/02; C12M 23/22; C12M 25/14; C12M 31/10

(57) ABSTRACT

Disclosed are methods and devices suitable for growing algae. In some embodiments, a device suitable for growing algae includes an algae growth-substrate assembly that comprises a pair of algae growth-substrates: an inwards-sloped substrate which directs liquid flowing on an upper surface thereof radially-inwards; and an outwards-sloped substrate which directs liquid flowing on an upper surface thereof radially-outwards, wherein the outwards-sloped substrate is located below and separated from the inwards-sloped substrate so that liquid from the inwards-sloped substrate falls onto the upper surface of the outwards-sloped substrate.

### RELATED APPLICATION

The present application gains priority from US provisional patent applications US 63/312,935 filed 23 Feb. 2022 which is included by reference as if fully set-forth herein.

### FIELD AND BACKGROUND OF THE INVENTION

The invention, in some embodiments, relates to the field of algaculture, waste processing and environmental sciences, and more particularly, but not exclusively, to methods and devices suitable for growing algae that in some embodiments are useful for wastewater processing and/or for carbon sequestration.

Commercial algaculture is important for producing products for many industries including food, cosmetics, pharmaceutical, chemical and fuel industries.

It is known to use algae to treat wastewater, for example, for processing blackwater to yield algae biomass and purified water, see for example, U.S. Pat. No. 9,771,287 and U.S. Pat. No. 11,041,142. Typically, the contents of the wastewater are used as nutrients for algal growth.

It is known to use algae in carbon sequestration (also known as CCS: carbon capture and storage), for example, from carbon-dioxide rich emissions from industries such as power plants.

Methods and devices for growing algae for algaculture, wastewater processing and/or carbon sequestration are sought.

### SUMMARY OF THE INVENTION

Some embodiments of the invention herein provide methods and devices for growing algae, that are in some aspects advantageous over those known in the art.

According to an aspect of some embodiments of the teachings herein, there is provided a device suitable for growing algae comprising:
a. a vessel having walls defining an inner volume, the inner volume having a top, a bottom, a height, a vertical axis, a vertical cross section including the vertical axis and a horizontal cross section perpendicular to the vertical axis;
b. a gas inlet located inside the inner volume functionally-associated with a gas conduit, so that a gas that passes through the gas conduit is released inside the inner volume through the gas inlet;
c. a liquid inlet located inside the inner volume functionally-associated with a liquid conduit, so that a liquid that passes through the liquid conduit is released inside the inner volume through the liquid inlet;
d. inside the inner volume and below the liquid inlet, an algae growth-substrate assembly so that liquid released inside the inner volume through the liquid inlet falls onto the algae growth-substrate assembly;
e. inside the inner volume and below the algae growth-substrate, a drain functionally-associated with a drain conduit so that a liquid in the inner volume that enters the drain passes into the drain conduit; and
e. inside the inner volume, an illumination assembly configured, when activated, to illuminate the inner volume from inside the inner volume with sufficient light for algal photosynthesis,
   wherein the algae growth-substrate assembly comprises a pair of algae growth-substrates:
an inwards-sloped substrate which directs liquid flowing on an upper surface thereof radially-inwards; and
an outwards-sloped substrate which directs liquid flowing on an upper surface thereof radially-outwards,
   wherein the outwards-sloped substrate is located below and separated from the inwards-sloped substrate so that liquid from the inwards-sloped substrate falls onto the upper surface of the outwards-sloped substrate. In some such embodiments, the illumination assembly comprises at least three physically-separated illumination units, each illumination unit comprising an elongated transparent container having a longitudinal axis and a horizontal cross section, each container containing a light source that, when activated, emits light that passes through walls of the transparent container to illuminate the inner volume, wherein each illumination unit is located inside the inner volume with the illumination unit longitudinal axis oriented within 10° of parallel with the vertical axis of the inner volume, wherein each illumination unit passes through a portion of the substrate assembly, does not intersect with the vertical axis of the inner volume and is devoid of contact with the walls of the vessel.

According to an aspect of some embodiments of the teachings herein, there is also provided a device suitable for growing algae comprising:
a. a vessel having walls defining an inner volume, the inner volume having a top, a bottom, a height, a vertical axis, a vertical cross section including the vertical axis and a horizontal cross section perpendicular to the vertical axis;
b. a gas inlet located inside the inner volume functionally-associated with a gas conduit, so that a gas that passes through the gas conduit is released inside the inner volume through the gas inlet;
c. a liquid inlet located inside the inner volume functionally-associated with a liquid conduit, so that a liquid that passes through the liquid conduit is released inside the inner volume through the liquid inlet;
d. inside the inner volume and below the liquid inlet, an algae growth-substrate assembly so that liquid released inside the inner volume through the liquid inlet falls onto the algae growth-substrate assembly;
e. inside the inner volume and below the algae growth-substrate, a drain functionally-associated with a drain conduit so that a liquid in the inner volume that enters the drain passes into the drain conduit; and
e. inside the inner volume, an illumination assembly configured, when activated, to illuminate the inner volume from inside the inner volume with sufficient light for algal photosynthesis,
   wherein the illumination assembly comprises at least three physically-separated illumination units, each illumination unit comprising an elongated transparent container having a longitudinal axis and a horizontal cross section, each container containing a light source that, when activated, emits light that passes through walls of the transparent container to illuminate the inner volume, wherein each illumination unit is located inside the inner volume with the longitudinal axis of the transparent container oriented within 10° of parallel with the vertical axis of the inner volume, wherein each illumination unit passes through a portion of the substrate assembly, does not intersect with the vertical axis of the inner volume and is devoid of contact with the walls of the vessel. In some such embodiments, the algae growth-substrate assembly comprises a pair of algae growth-substrates:
an inwards-sloped substrate which directs liquid flowing on an upper surface thereof radially-inwards; and
an outwards-sloped substrate which directs liquid flowing on an upper surface thereof radially-outwards,
   wherein the outwards-sloped substrate is located below and separated from the inwards- sloped substrate so that liquid from the inwards-sloped substrate falls onto the upper surface of the outwards-sloped substrate.

In some embodiments, during use of a device, the vertical axis of the inner volume is within about 20° of parallel with the gravity vector.

In some embodiments, the height of the inner volume is not less than about 100 cm and not more than about 500 cm.

In some embodiments, the horizontal cross section of the inner volume has an average area of not less than about 310 cm² (equivalent to that a 10 cm radius circle) and not more than about 630 cm² (equivalent to that of a 25 cm radius circle).

In some embodiments, the volume of the inner volume is not less than about 40 liters and not more than about 400 liters.

In some embodiments, the horizontal cross section of the inner volume is a closed curve. In some such embodiments, the inner volume has a shape selected from the group consisting of tubular, truncated conical and conical.

In some embodiments, the inner volume is cylindrical. In some such embodiments, the vessel comprises a cylindrical pipe that defines side walls of the vessel, an upper wall capping a top end of the pipe and a bottom wall capping a bottom end of the pipe.

In some embodiments, the illumination assembly comprises at least one illumination unit, each illumination unit including a transparent container located inside the inner volume, the transparent container containing a light source that, when activated, emits light that passes through walls of the transparent container.to illuminate the inner volume from inside the inner volume. In some such embodiments, in at least one illumination unit, a light source is integrally forned with a respective transparent container. Alternatively or additionally, in some such embodiments, in at least one illumination unit, a light source is separable from a respective transparent container.

In some embodiments, a distance of a longitudinal axis of at least one of the illumination units of the illumination assembly from the vertical axis of the inner volume is not less than about 20% and note more than about 80% of the distance from the vertical axis of the inner volume to an inner surface of the walls of the vessel,

In some embodiments, the walls of the vessel comprise a side wall. In some such embodments, the side wall comprises a tube. In some such embodiments, in some embodiments the illumination assembly is not attached to the side wall. Additionally or alternatively, in some such embodiments, the algae growth-substrate assembly is not attached to the side wall.

In some embodiments, the algae growth-substrate assembly is attached to the illumination assembly. In some such embodiments, the device is configured to allow removal of the side wall while the algae growth-substrate assembly remains attached to the illumination assembly.

In some embodiments, the algae growth-substrate assembly comprises at least two pairs of algae growth-substrates (as described above) at different heights in the inner volume, where a given pair of algae growth-substrates is positioned above and/or below at least one other pair of algae growth-substrates so that an inwards-sloped substrate of a lower pair is located below and separated from an outwards-sloped substrate of a higher pair so that liquid from the outwards-sloped substrate of the higher pair falls onto an upper surface of the inwards-sloped substrate of a lower pair.

In some embodiments, at least one substrate is impermeable to water.

Additionally or alternatively, in some embodiments at least one substrate is permeable to water. In some such embodiments, a permeable substrate is selected from the group consisting of a net and textile.

According to an aspect of some embodiments of the teachings herein, there is provided a waste processing system comprising at least two devices as described herein.

In some embodiments, at least two of the at least two devices are arranged in series so that a gas outlet of a first of the two devices is in fluid communication with a gas inlet of a second of the two devices so that gas released from an inner volume of the first device is processed in an inner volume of the second device.

Additionally or alternatively, in some embodiments at least two of the at least two devices are arranged in series so that a drain of a first of the two devices is in fluid communication with a liquid inlet of a second of the two devices so that liquid exiting the inner volume of the first device is processed in an inner volume of the second device.

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

In the Figures:

FIGS. 1A-1C depict an embodiment of a device as described herein, in vertical cross section (FIG. 1A), horizontal cross section along A-A (FIG. 1B) and partial vertical cross section showing a presumed flow of liquid in the device (FIG. 1C);

FIGS. 2A-2D depict an embodiment of a device as described herein, in vertical cross section (FIG. 2A), horizontal cross section along A-A (FIG. 2B), partial side view from direction B (FIG. 2C) and partial vertical cross section showing a presumed flow of liquid in the device (FIG. 2D);

FIGS. 3A-3B depict an embodiment of a device as described herein, in vertical cross section (FIG. 3A) and partial vertical cross section showing a presumed flow of water in the device (FIG. 3B);

FIG. 4 depicts a variant of the device depicted in FIGS. 3 in partial vertical cross section showing a presumed flow of water in the device;

FIGS. 5A-5F each depict embodiments of a device as described herein in horizontal cross section, each FIG. 5 showing an embodiment with a different illumination assembly;

FIG. 6 depicts an embodiment of a device as described having a square horizontal cross section; and

FIGS. 7A and 7B schematically depict an embodiment of a device as described herein having a trapeze vertical cross section.

### DESCRIPTION OF SOME EMBODIMENTS OF THE INVENTION

Some embodiments of the invention herein provide methods and devices for growing algae, that are in some aspects advantageous over those known in the art.

The principles, uses and implementations of the teachings of the invention may be better understood with reference to the accompanying description and figures. Upon perusal of the description and figures present herein, one skilled in the art is able to implement the teachings of the invention without undue effort or experimentation. In the figures, like reference numerals refer to like parts throughout.

Before explaining at least one embodiment of the invention in detail, it is to be understood that the invention is not necessarily limited in its application to the details of construction and the arrangement of the components and/or methods set forth herein. The invention is capable of other embodiments or of being practiced or carried out in various ways. The phraseology and terminology employed herein are for descriptive purpose and should not be regarded as limiting.

In some embodiments, the teachings herein provide a device suitable for growing algae where the device can be used for algaculture to provide algal biomass for harvest and/or to treat wastewater and/or for carbon sequestration. In some embodiments, the device is compact, simple and cheap to build from common materials, which is particularly important for wastewater processing and carbon sequestration where low costs are critical. In some embodiments, the device is simple and economical to operate and/or maintain, even in remote areas. In some embodiments, the device has a small footprint, allowing the use of the device in small places and even secured to a wall, smokestack or chimney. In some embodiments, the device allows relatively precise control of operation parameters such as the inflow of nutrient-containing water, the inflow of carbon dioxide containing gas and the intensity/schedule of light for photosynthesis. Such control allows for efficient use of the device to achieve one or more goals of algae yield and/or wastewater purification and/or carbon sequestration. In some embodiments, multiple devices can be used together (in series or in parallel) to increase processing capacity.

In some embodiments, a device as described herein is a photobioreactor which receives nutrient-rich water (including wastewater) and a gas comprising carbon-dioxide and produces algal biomass for harvest.

Additionally or alternatively, in some embodiments, a device as described herein is a carbon-sequestering device, for example, a device receives the exhaust of a carbon dioxide emitter (such as a fossil-fuel burning power plant) and at least some of the carbon dioxide in the exhaust is converted to algal biomass instead of being released into the atmosphere.

Additionally or alternatively, in some embodiments, a device as described herein is a wastewater treatment device, for example, a device converts received wastewater into algal biomass and clean water.

According to an aspect of some embodiments of the teachings herein, there is provided a device suitable for growing algae comprising:
a. a vessel having walls defining an inner volume, the inner volume having a top, a bottom, a height, a vertical axis, a vertical cross section including the vertical axis and a horizontal cross section perpendicular to the vertical axis;
b. a gas inlet located inside the inner volume functionally-associated with a gas conduit, so that a gas that passes through the gas conduit is released inside the inner volume through the gas inlet;
c. a liquid inlet located inside the inner volume functionally-associated with a liquid conduit. so that a liquid that passes through the liquid conduit is released inside the inner volume through the liquid inlet;
d. inside the inner volume and below the liquid inlet, an algae growth-substrate assembly so that liquid released inside the inner volume through the liquid inlet falls onto the algae growth-substrate assembly;
e. inside the inner volume and below the algae growth-substrate, a drain functionally-associated with a drain conduit so that a liquid in the inner volume that enters the drain passes into the drain conduit; and
e. inside the inner volume, an illumination assembly configured, when activated to illuminate the inner volume from inside the inner volume with sufficient light for algal photosynthesis;
   wherein the algae growth-substrate assembly comprises a pair of algae growth-substrates:
an inwards-sloped substrate which directs liquid flowing on an upper surface thereof radially-inwards; and
an outwards-sloped substrate which directs liquid flowing on an upper surface thereof radially-outwards,
   wherein the outwards-sloped substrate is located below and separated from the inwards-sloped substrate so that liquid from the inwards-sloped substrate falls onto the upper surface of the outwards-sloped substrate. In some embodiments, the illumination assembly comprises at least three physically-separated illumination units, each illumination unit comprising an elongated transparent container having a longitudinal axis and a horizontal cross section (perpendicular to the longitudinal axis), each container containing a light source that, when activated emits light that passes through walls of the transparent container to illuminate the inner volume, wherein each illumination unit of the illumination assembly is located inside the inner volume with the longitudinal axis oriented within 10° of parallel with the vertical axis of the inner volume, wherein each illumination unit passes through a portion of the substrate assembly, does not intersect with the vertical axis of the inner volume and is devoid of contact with walls of the vessel.

During operation of the device, there is sessile algae attached to the substrates of the substrate assembly.

During operation of the device, a liquid, preferably with nutrients for algae such as wastewater, is introduced into the inner volume through the liquid inlet to fall onto the algae growth-substrate assembly while a gas comprising carbon dioxide is introduced into the inner volume through the gas inlet and the illumination assembly is activated to provide light for photosynthesis inside the inner volume. Nutrients (if present) in the liquid that flows down the substrate assembly and carbon dioxide are used by photosynthesizing sessile algae (e.g., benthic algae) growing on the substrates of the substrate assembly to produce algal biomass.

One of the pair of algae growth-substrates is an inwards-sloped substrate which directs liquid flowing on an upper surface thereof radially-inwards, for example towards the vertical axis. In some embodiments, the inwards-sloped substrate can be considered a bowl or funnel oriented with the wide opening upwards which causes liquid that falls onto and flows along the upper suface of the substrate radially-inwards.

Another one of the pair of algae growth-substrates is an outwards-sloped substrate which directs liquid flowing on an upper surface thereof radially-outwards, in some embodiments away from the vertical axis. In some embodiments, the outwards-sloped substrate can be considered a bowl with the wide opening downwards which causes liquid that falls onto and flows along the upper suface of the substrate radially-outwards.

As will be described in greater detail below, a substrate assembly that comprises multiple separated algae growth-substrates inside the inner volume gives the device a particular high substrate surface area for the anchoring and growth of a comparatively large amount of sessile algae relative to the size of the inner volume of the device. The positioning of one substrate above the other allows the device to have a compact footprint. The combination of an inwards-sloped substrate which directs liquid flowing on an upper surface thereof radially inwards, which liquid falls onto the upper surface of an outwards-sloped substrate which directs liquid flowing on an upper surface thereof radially outwards provides efficient passive (resulting from gravity) mixing of the liquid inside the inner volume. In preferred embodiments, such a substrate assembly assists in:

illumination of most or all of the liquid by light from the illumination assembly, release of oxygen produced by algal photosynthesis from the liquid; and
provides the liquid with a high surface area thereby allowing efficient absorption of carbon dioxide by the liquid from the gas inside the inner volume of the vessel of the device.
   Further, in preferred embodiments, some or all of the substrates are water permeable so that some of the liquid flows through such a water permeable substrate and then flows along the bottom surface of the substrate and/or drops down as droplets or narrow streams. As a result, mixing and illumination of the liquid are even more efficient and the surface area of the liquid for release of oxygen and absorption of carbon dioxide is even greater.

During use, a device according to the teachings herein is placed so that the vertical axis of the inner volume is within about 20° of parallel to the gravity vector (as depicted in the Figures) so that the top of the inner volume is above the bottom of the inner volume. In some embodiments, the vertical axis is within about 15°, within about 10° and even within about 5° of parallel to the gravity vector.

According to an aspect of some embodiments of the teachings herein, there is also provided a device suitable for growing algae comprising:
a. a vessel having walls defining an inner volume, the inner volume having a top, a bottom, a height, a vertical axis, a vertical cross section including the vertical axis and a horizontal cross section perpendicular to the vertical axis;
b. a gas inlet located inside the inner volume functionally-associated with a gas conduit, so that a gas that passes through the gas conduit is released inside the inner volume through the gas inlet;
c. a liquid inlet located inside the inner volume functionally-associated with a liquid conduit, so that a liquid that passes through the liquid conduit is released inside the inner volume through the liquid inlet;
d. inside the inner volume and below the liquid inlet, an algae growth-substrate assembly so that liquid released inside the inner volume through the liquid inlet falls onto the algae growth-substrate assembly;
e. inside the inner volume and below the algae growth-substrate, a drain functionally-associated with a drain conduit so that a liquid in the inner volume that enters the drain passes into the drain conduit; and
e. inside the inner volume, an illumination assembly configured, when activated to illuminate the inner volume from inside the inner volume with sufficient light for algal photosynthesis,
   wherein the illumination assembly comprises at least three physically-separated illumination units, each illumination unit comprising an elongated transparent container having a longitudinal axis and a horizontal cross section, each transparent container containing a light source that, when activated, emits light that passes through walls of the transparent container to illuminate the inner volume, wherein each illumination unit is located inside the inner volume with the longitudinal axis of the container oriented within 10° of parallel with the vertical axis of the inner volume, wherein each illumination unit passes through a portion of the substrate assembly, does not intersect with the vertical axis of the inner volume and is devoid of contact with the walls of the vessel. In some embodiments, the algae growth-substrate assembly comprises a pair of algae growth-substrates as described above:
an inwards-sloped substrate which directs liquid flowing on an upper surface thereof radially-inwards; and
an outwards-sloped substrate which directs liquid flowing on an upper surface thereof radially-outwards,
   wherein the outwards-sloped substrate is located below and separated from the inwards-sloped substrate so that liquid from the inwards-sloped substrate falls onto the upper surface of the outwards-sloped substrate.

### Vessel

As noted above, in some embodiments a device according to the teachings herein comprises a vessel having walls defining an inner volume, the inner volume having a top, a bottom, a height, a vertical axis, a vertical cross section including the vertical axis and a horizontal cross section perpendicular to the vertical axis.

### Inner Volume

The inner volume is any suitable size.

In some typical embodiments, the height of the inner volume (dimension parallel to the vertical axis) is not less than about 100 cm and not more about 500 cm. In some such embodiments, the height of the inner volume is not less than about 150 cm. Additionally or alternatively, in some embodiments the height of the inner volume is not more than about 400 cm and not more than about 350 cm.

In some embodiments, the horizontal cross section has an average area of not less than about 310 cm² (equivalent to a 10 cm radius circle) and not more than about 630 cm² (equivalent to a 25 cm radius circle).

In some embodiments, the volume of the inner volume is not less than about 40 liters and not more than about 400 liters. In some such embodiments, the inner volume is not more than about 300 liter. Exemplary embodiments include:
a device having a 1.5 m high inner volume with a circular horizontal cross section having a radius of 10 cm and a volume of about 48 liter;
a device having a 3 m high inner volume with a circular horizontal cross section having a radius of 10 cm and a volume of about 94 liter; and
a device having a 2 m high inner volume with a circular horizontal cross section having a radius of 20 cm and a volume of about 260 liter.

The inner volume is of any suitable shape.

In some embodiments, the horizontal cross section of the inner volume is a closed curve like a circle or oval: such embodiments are devoid of internal edges that can accumulate dirt, grime and pathogens, except at the top and bottom which internal edges are limited in size and relatively easy to clean.

In some embodiments, the shape of the horizontal cross section is the same along the height dimension. Alternatively, in some embodiments the shape of the horizontal cross section changes along the height dimension.

In some embodiments, the size (i.e., cross sectional area) of the horizontal cross section is constant along the height dimension and the side walls are parallel to the vertical axis. Alternatively, in some embodiments the size of the horizontal cross section is not constant along the height dimension and the side walls are not parallel to the vertical axis, in preferred embodiments the size of the horizontal cross section decreasing closer to the top.

In some preferred embodiments, the inner volume is cylindrical, having a circular horizontal cross section of the same size along the height dimension and side walls that are parallel to the vertical axis.

In some alternative preferred embodiments, the inner volume is conical or truncated conical, having a circular horizontal cross section which decreases in size closer to the top, and straight side walls that are not parallel to the vertical axis.

### Walls

The walls of the vessel are of any suitable material. In some preferred embodiments are made of a plastic. In some embodiments, the walls of the vessel are made of a plastic selected from the group consisting of PVC (polyvinyl chloride), CPVC (chlorinated polyvinyl chloride), ABS (acrylonitrile butadiene styrene), HDPE (high density polyethylene), PEX (crosslinked polyethylene) and PMMA (polymethyl methacrylate).

In some embodiments, the walls comprise a side wall, a top wall and a bottom wall. In some such embodiments, the side wall is a tube (e.g., having a closed curve cross section, preferably a circular cross section) where the top wall and bottom wall can be considered as caps at the ends of the tube. In some such embodiments, the side wall is a plastic pipe as is known in the art of water conduits such as standard PVC or CPVC water pipes having any standard diameter (6″, 8″, 10″, 12″, 16″) as these are readily available, physically sturdy, and resistant to the conditions in which a device is to be operated. Accordingly, in some embodiments, the vessel comprises a cylindrical pipe that defines side walls of the vessel, an upper wall capping a top end of the pipe and a bottom wall capping a bottom end of the pipe.

Wall transparency

In some embodiments the walls are transparent or translucent to light so that ambient light can pass through the walls into the inner volume. In such embodiments, if the device is located outside then under certain conditions ambient light (e.g., from the sun or from lamps) is sufficient to support photosynthesis of algae inside the inner volume. Disadvantages of such embodiments include that such light encourages growth of algae on the inner side of the walls rather than on the substrates and/or that solar UV can potentially damage internal components or suppress algae growth. Some such embodiments are designed for operation in a place with little or no ambient light, for example inside a building, so that the illumination assembly is the primary or, more preferably, the exclusive source of light that supports photosynthesis in the inner volume.

In preferred embodiments, the primary and more preferably exclusive source of light that supports photosynthesis in the inner volume is the illumination assembly. Such embodiments allow illumination according to a desired schedule at a desired intensity (e.g., continuously or when there is processing need to increase photosynthesis). In some preferred embodiments, when the device is operated, ambient light (visible and UV) is prevented from passing through the walls of the vessel into the inner volume.

In some embodiments the walls are transparent or translucent to visible but opaque to UV light, that is to say, the walls are configured (e.g., due to the wall material or a coating) so that less than 1% of ambient UV light can pass through the walls of the vessel into the inner volume. Such embodiments are useful in preventing UV damage to device components that are located inside the inner volume and useful in preventing algae growth-suppression by UV light.

In some embodiments the walls are opaque to visible and to UV light, that is to say, the walls are configured so that less than 1% of ambient visible and UV light can pass through the walls of the vessel into the inner volume, in some embodiments less than 0.5% and in some embodiments even less than 0.1% of ambient visible and UV light.

In some such embodiments, the walls are made of an opaque material, for example, the walls are made of standard PVC and CPVC water pipes that are opaque to UV and visible light. Additionally or alternatively, in some such embodiments, the device comprises a light shield positioned outside of the vessel (e.g., as a cover, an envelope, a sleeve surrounding the walls of the vessel), the light shield opaque to UV and visible light. Additionally or alternatively, in some such embodiments, the device comprises an opaque liner coating or covering the internal surface of the walls of the inner volume, the liner opaque to UV and visible light.

In some embodiments, the walls of the inner volume are mirrored so that light from the illumination assembly that impinges on the walls is reflected back into the inner volume.

### Gas Inlet

As noted above, in some embodiments a device according to the teachings herein comprises a gas inlet located inside the inner volume, functionally-associated with a gas conduit so that a gas that passes through the gas conduit is released inside the inner volume through the gas inlet. As noted above, during operation of the device, the function of the gas inlet is to allow introduction of carbon dioxide required for algal photosynthesis.

The gas inlet is preferably located below the algae growth-substrate assembly, preferably close to the bottom of the inner volume, to avoid the possible of the formation of a carbon dioxide-poor volume in parts of the growth substrate assembly.

In some embodiments, the gas inlet is configured to avoid entry of liquid that falls from the substrate assembly into the gas inlet.

In some embodiments, the device is configured for or operated under conditions that the bottom of the inner volume is a reservoir of liquid having a depth. In some such embodiments, the gas inlet is elevated from the bottom of the inner volume to be higher than the depth. In some such embodiments, the gas inlet is configured to also operate when submerged under liquid in the reservoir so that gas from the gas inlet bubbles through the liquid in the reservoir.

In some embodiments, the gas conduit passes through a side wall of the device. In preferred embodiments, the gas conduit passes through a bottom wall of the device and not a side wall of the device.

In some embodiments, a device comprises a single gas inlet functionally-associated with a single gas conduit. In some embodiments, a device comprises a single gas inlet functionally-associated with at least two gas conduits. In some embodiments, a device comprises at least two gas inlets, at least one functionally-associated with a single gas conduit. In some embodiments, a device comprises at least two gas inlets, at least one functionally-associated with at least two gas conduits.

In some embodiments (for example, some embodiments implemented for wastewater processing), the gas conduit is functionally-associated with a pump to drive ambient air into the gas conduit and then to be released inside the inner volume through the gas inlet.

In some embodiments (for example, some embodiments implemented for carbon sequestration), the gas conduit is functionally-associated with an exhaust (e.g., of a device, process or system, such as an industrial device, process or system for example, a flue, or of an internal combustion engine) so that exhaust gases from the exhaust is released inside the inner volume through the gas inlet.

In some embodiments, the gas that is released inside the inner volume through the gas inlet includes one or more pollutants that are at least partially removed from the gas in the inner volume by interaction with algae, in some embodiments the pollutants being used as nutrients by the algae. In some such embodiments, at least one pollutant is selected from the group consisting of ammonia, NOx and SOx.

Typically, as a result of algal photosynthesis, the carbon dioxide content of gas released inside the inner volume through the gas inlet is higher than the carbon dioxide content of gas that exits the inner volume, for example, via a gas outlet.

In some embodiments, the gas released inside the inner volume through the gas inlet is ambient atmospheric air (typically atmospheric air that is driven into the gas conduit by a pump) having a carbon dioxide content of about 400 ppm. In such embodiments, the gas that exits the device typically has a carbon dioxide content less than that of the ambient atmospheric air.

In some embodiments, the gas released inside the inner volume through the gas inlet has a carbon dioxide content substantially higher than that of atmospheric air, for example in some embodiments where the released gas is exhaust and/or in some embodiments when the device is used for carbon sequestration. In such embodiments, the device is preferably operated so that the gas that exits the device has a carbon dioxide content that is equal or less than of ambient atmospheric air, for example, by release of a required amount of nutrients inside the inner volume through the liquid inlet and/or by activating the illumination assembly to illuminate the inner volume with sufficient light.

### Liquid Inlet

As noted above, in some embodiments a device according to the teachings herein comprises a liquid inlet located inside the inner volume. The liquid inlet is located above the algae growth substate assembly and is functionally-associated with a liquid conduit so that a liquid that passes through the liquid conduit is released inside the inner volume through the liquid inlet and falls onto the algae growth-substrate assembly.

During operation of the device in some embodiments where the device is used for wastewater treatment, growth of algal biomass and/or carbon sequestration, the liquid that passes through the liquid conduit and into the inner volume through the liquid inlet is wastewater to be treated. In some embodiments where the device is used for the growth of algal biomass and/or carbon sequestration, the liquid that passes through the liquid conduit and into the inner volume through the liquid inlet can be water, preferably water that includes nutrients required for algal growth.

In some embodiments, the device comprises a pump functionally-associated with the liquid conduit to drive liquid from a source of liquid into the inner volume through the recycling conduit and the liquid inlet.

In some embodiments, the liquid conduit passes through a side wall of the device. In preferred embodiments, the liquid conduit passes through a bottom wall of the device and not a side wall of the device.

In some embodiments, a device comprises a single liquid inlet functionally-associated with a single liquid conduit. In some embodiments, a device comprises a single liquid inlet functionally-associated with at least two liquid conduits. In some embodiments, a device comprises at least two liquid inlets, at least one functionally-associated with a single liquid conduit. In some embodiments, a device comprises at least two liquid inlets, at least one functionally-associated with at least two liquid conduits.

In some embodiments, the device comprises a recycling conduit, a liquid conduit provides fluid communication between the drain and the liquid inlet, allowing liquid that exits the inner volume through the drain to be reintroduced into the inner volume through the recycling conduit and a liquid inlet. In some such embodiments, the recycling conduit is functionally-associated with a pump to drive liquid from the drain into the inner volume through the recycling conduit and the liquid inlet. Embodiments comprising a recycling conduit are particularly suitable for wastewater processing.

In some embodiments where the uppermost substrate of the algae growth-substrate assembly is an outwards-sloped substrate which directs liquid flowing on an upper surface thereof radially-outwards, the device comprises a single liquid inlet which releases liquid from a single location, e.g., as a single stream or spray. In some such embodiments, a single stream is released towards the apex of the upper-most substrate so that the liquid is radially-distributed while flowing on the upper surface of the uppermost substrate.

In preferred embodiments, and especially in embodiments where the upper-most substrate of the algae growth-substrate assembly is an inwards-sloped substrate which directs liquid flowing on an upper surface thereof radially-inwards, the device comprises more than one liquid inlet so that liquid is released inside the inner volume at multiple locations, e.g., as streams and/or sprays. In some such embodiments, the device comprises not least than 4, not less than 6 and even not less than 8 liquid inlets. In some such embodiments, a liquid conduit is a manifold having multiple liquid inlet inlet-bearing branches. In some such embodiments, a terminal portion of the liquid condit is a ring having multiple liquid inlets on the ring.

### Drain

As noted above, in some embodiments a device according to the teachings herein comprises, inside the inner volume and below the algae growth-substrate, a drain functionally-associated with a drain conduit so that a liquid in the inner volume that enters the drain passes into the drain conduit.

In some embodiments, the drain is a passive drain positioned at a desired height from the bottom of the inner volume. When the water level in the inner volume reaches the height of the drain, the water enters the drain and is removed from the inner volume through the drain conduit.

In some embodiments, the drain is an active drain that is to say, has a mechanism that detects a level of liquid in the inner volume and, when the level exceeds a threshold, opens the active drain to allow liquid to drain from the inner volume. As noted above, one suitable such mechanism includes a ballcock as known in the art.

During operation of the device, some algae disconnects from the substrates and falls towards the bottom of the inner volume. In some such embodiments, disconnected algae enters the drain and the drain conduit. In some such embodiments, the drain conduit directs the disconnected algae together with drained water to be discarded. In some embodiments, the drain conduit is functionally-associated with an algae harvester (e.g., an algae-harvesting conveyor belt or drum as known in the art) to recover disconnected algae from the water. In some embodiments, such passive harvesting of algae (of algae that spontaneously falls from the substrate and exits the outlet) is an advantageous feature that, in some embodiments, occurs efficiently and spontaneously as a result of the particular configuration of the substrate assembly as described herein.

In some embodiments, the drain conduit is functionally-associated with a mechanism to kill algae spores, for example, a heater or UV lamp.

### Illumination Assembly

As noted above, in some embodiments a device according to the teachings herein comprises an illumination assembly configured, when activated, to illuminate the inner volume from inside the inner volume with sufficient light for algal photosynthesis.

In the art, it is known to use light from an illumination assembly as an adjunct to free solar light for algal photosynthesis. In contrast, according to preferred embodiments of the teachings herein, the illumination assembly that is located inside the inner volume is preferably the primary, and more preferably the exclusive source of light for algal photosynthesis inside the inner volume. To this end and as discussed herein, in some embodiments, some, and even all, of the walls of the chamber are opaque so that no ambient light suitable for algal photosynthesis enters the inner chamber.

Some embodiments of the device have one or more of advantages resulting from limiting the use of solar light for algal photosynthesis including:
allowing the use of non-transparent materials for the construction of the walls of the vessel of the device, including standard pipes known in the art of sewage and water transport, such as PVC and CPVC pipes;
reducing or completely avoiding damage to plastic components of the device by UV light from the sun;
reducing the extent of the growth of algae on the inner surface of the vessel walls (which is encouraged by solar illumination through a transparent walls as the inner surface is where the illumination is most intense) which growth reduces the performance of the device by reducing the amount of light that penetrates into the inner volume;
encouraging algal growth on the substrates of the substrate assembly, including areas of the substrates that are further from the wall of the container; and
allowing control of the intensity and duration of the illumination, and therefore the rate and timing of algal photosynthesis according to processing needs, independently of the time of day, season and weather.

In some embodiments, the illumination assembly includes a single illumination unit which is located inside the inner volume and configured, when activated, to illuminate the inner volume from inside the inner volume with sufficient light for algal photosynthesis. In some preferred embodiments, the illumination includes at least two illumination units that are physically separated one from the other, each illumination unit located inside the inner volume and configured, when activated, to illuminate the inner volume from inside the inner volume with sufficient light for algal photosynthesis.

In some embodiments, each illumination unit of the illumination assembly includes a transparent container located inside the inner volume, the transparent container containing a light source that, when activated, emits light that passes through the walls of the transparent container to illuminate the inner volume from inside the inner volume.

Light source

Any suitable light source can be used for implementing the teachings herein. LEDs (light-emitting diodes) are preferred as these are readily commercially-available, can be shaped and dimensioned as desired, emit a suitable intensity of light, do not emit UV light and are energy efficient. In some embodiments, the LEDs are submersible LEDS that can be activated to emit light even when immersed in water.

In some embodiments, the illumination assembly is configured to emit only red light, e.g., comprises only red LEDs, especially embodiments where it is useful to prefer algae that preferentially uses red light for photosynthesis, or to discourage growth of algae that preferentially uses light other than red for photosynthesis.

In some embodiments, the illumination assembly is configured to emit only blue light, e.g., comprises only blue LEDs, especially embodiments where it is useful to prefer algae that preferentially uses green blue for photosynthesis, or to discourage growth of algae that preferentially uses light other than blue for photosynthesis.

In some embodiments, the illumination assembly is configured to emit only blue and red light, e.g., comprises only blue and red LEDs. Such embodiments are currently believed to be most suitable for carbon sequestration and wastewater treatment.

In some embodiments, LED-strips are used as the light source, for example, LED strips that require not less than 10 W per meter, not less than 12 W per meter and even LED strips that require not less than 15 W per meter when providing the maximal intensity. Such LED strips typically comprise 300 individual LEDs per meter of backing, for example 300 red LEDs per meter of backing, 300 blue LEDs per meter of backing, and 150 red and 150 blue LEDs per meter of backing.

In some embodiments, the device is configured to allow varying the intensity of light that the illumination assembly. In some such embodiments, varying the intensity of light comprises changing the number of individual light sources (e.g., LEDs) are activated. Additionally or alternatively, in some embodiments varying the intensity of light comprises changing the characteristics of the electrical current that is supplied to the light source (e.g., changing the current that flows through a light source such as LED (for example, changing the value of the current in A, alternately stopping and starting the current thereby causing LED light sources to “blink”). A person having ordinary skill in the art of LEDs is able to implement such a feature with no inventive effort.

### Transparent Container

The transparent container is a container that holds the light source, such as LEDs, for example LEDs on a LED strip, inside the inner volume, protects the light source from contact with the liquid contents of the inner volume that can potentially damage or degrade the light source.

In some embodiments, the light source is integrally formed with the transparent container, for example, on or more LED strips embedded inside a transparent polymer, for example a transparent polymer rod. For example, in some embodiments a illumination unit of an illumination assembly is a 20 mm solid transparent polymer rod, preferably at least as long as the height of the inner volume, with one or more LED strips embedded therein.

In preferred embodiments, the light source is separable from the transparent container so that the light source can be removed from the transparent container for maintenance or replacement, preferably without dissassembling the device. In some embodiments, a illumination unit of an illumination assembly is a transparent polymer rod with at least one bore, where the light source is located inside one or more bores. In embodiments with a single bore, the rod is a tube. In preferred such embodiments, the light source can be taken out and placed back in a bore without moving the polymer rod relative to the device. For example, in some embodiments a illumination unit of an illumination assembly is a 25 mm polymer tube having 5 mm thick walls and a 15 mm diameter bore, preferably at least as long as the height of the inner volume, with one or more LED strips located inside the bore.

The material from which the container is made is any suitable material or combination of materials. A suitable material is transparent to the wavelengths which are to be used to support algal photosynthesis, resistant to the conditions inside the inner volume and is preferably resistant to attachment by algae. Suitable materials include, for example, glass, crystal, ceramic, aluminum oxynitride (“transparent aluminum”) and polymers. In preferred emboidments, the container is a polymer. In some embodiments, the polymer is selected from the group consisting of PMMA, PVC, CPVC and polycarbonate. In some embodiments, preferred materials are PMMA and CPVC. In some embodiments, the outer surface of a container (the surface which is exposed to the conditions inside is inner volume) is treated to reduce attachment of algae thereto, for example, includes an anti-algae coating, e.g., by Blocksil® Ltd., Lichfield, United Kingdom.

### Location of the Illumination Assembly

The one or more illumination units of the illumination assembly are located in any suitable location inside the inner volume.

### Peripheral Illumination Assembly Illumination Unit

In some embodiments, an only illumination unit or one of at least two illumination units of the illumination assembly is located at the periphery of the inner volume and, when activated, provides light from the periphery towards the center of the inner volume. In some such embodiments, the container is a solid transparent component with the light sources integrally formed therewith (e.g., embedded therein) wherein one of the sides of the container contacts the inner surface of the vessel walls, for example, the container is a sheet (contacting the inner surface of flat walls of a vessel) or a tube (inside the inner volume and concentric with the walls of a tubular vessel). Alternatively, in some such embodiments, the container defines a space between the inner walls of the vessel and the portion of the inner volume wherein the substrate assembly is located, where the light sources are located inside the space.

### Central Illumination Assembly Illumination Unit

Additionally or alternatively, in some embodiment an only illumination unit or one of at least two illumination units of the illumination assembly is centrally located inside the inner volume along the vertical axis thereof and, when activated, provides light from the center of the inner volume outwards towards the walls of the vessel. In preferred embodiments, the container is an elongated container (such as a rod or tube) having a longitudinal axis and a horizontal cross section, the longitudinal axis oriented within 10° (more preferably within 5° and even more preferably within 2°) of parallel of the vertical axis of the inner volume. The horizontal cross section of the elongated container is of any suitable shape, in some embodiments selected from the group consisting of circular, square, pentagonal, hexagonal and rectangular. In some such embodiments, the light sources are integrally formed with the elongated container (e.g., embedded therein),. Alternatively, in some such embodiments, the elongated container includes one or more axial bores, with light sources located inside one or more of the axial bores. In such embodiments where the elongated container hasa single bore, the elongated container is a tube such as a pipe. The axial length of such a central illumination unit is preferably not less than the axial height of the algae growth-substrate assembly and, preferably, not less than the axial height of the inner volume.

### Multiple Off-Center Elongated Illumination Assembly Illumination Units

Additionally or alternatively, (and as briefly described above) in some preferred embodiments the illumination assembly comprises at least three physically-separated illumination units, each illumination unit comprising an elongated transparent container having a longitudinal axis and a horizontal cross section, each elongated container containing a light source that, when activated, emits light that passes through walls of the transparent container to illuminate the inner volume, wherein each illumination unit is located inside the inner volume with the longitudinal axis oriented within 10° (more preferably within 5° and even more preferably within) 2° of parallel with the vertical axis of the inner volume, wherein each illumination unit passes through a portion of the substrate assembly, does not intersect with the vertical axis of the inner volume and is devoid of contact with the walls of the vessel that define the inner volume.

The axial length of such elongated illumination units is preferably not less than the axial height of the algae growth-substrate assembly and, preferably, not less than the axial height of the inner volume.

The horizontal cross section of the elongated transparent containers is of any suitable shape, in some embodiments selected from the group consisting of circular, square, pentagonal, hexagonal and rectangular. In preferred embodiments, the elongated transparent containers are transparent tubes, e.g., of PMMA having a circular horizontal cross section.

In some embodiments, the light from such an illumination assembly illuminates the substrates of substrate assembly relatively homogeneously, including both the bottom and the top sides of the substrates, with few if any unilluminated portions,

In some embodiments, the illumination unit assembly comprises only three such illumination units comprising an elongated transparent container. In some embodiments, the illumination unit assembly comprises at least four, at least five, at least six and even at least eight such illumination units comprising an elongated transparent container: in some embodiments, a number of such illumination units provides illumination of the substrates with an even greater degree of homogeneity.

In some embodiments, the distance of the longitudinal axis of at least one, preferably all of the at least three, illumination units from the vertical axis of the inner volume is not less than about 20% and note more than about 80% of the distance from the vertical axis of the inner volume to the inner surface of the walls of the vessel. In some such embodiments the distance of the longitudinal axis of at least one, preferably all of the at least three, illumination units from the vertical axis of the inner volume is not less than about 30% and even not less than about 40% of the distance from the vertical axis of the inner volume to the inner surface of the walls of the vessel. In some such embodiments the distance of the longitudinal axis of at least one, preferably all of the at least three, illumination units from the vertical axis of the inner volume is not more than about 70% and even not less than about 60% of the distance from the vertical axis of the inner volume to the inner surface of the walls of the vessel.

### Parts of the Illumination Assembly are a Structural Component

A device according to the teachings herein is assembled form the components in any suitable way including by suitably attaching, permanently or reversibly, any two components. Such assembly can be performed by a person having ordinary skill in the art upon persual of the description and figures without requiring inventive effort.

As noted above, in some embodiments, in some embodiments, the walls of the vessel of device comprise a side wall, in some such embodiments the side wall is a tube (such as a pipe). In some such embodiments, the walls of the vessel further comprise a bottom wall and/or a top wall. In some such embodiments, the side wall is a tube and the top wall and bottom wall can be considered as caps at the ends of the tube.

In some such embodiments, the gas inlet is not attached to a side wall. In some such embodiments, the gas inlet is attached to a bottom wall.

Additionally or alternatively, in some such embodiments, the drain is not attached to a side wall. In some such embodiments, the drain is attached to or is a component of a bottom wall.

Additionally or alternatively, in some such embodiments, the liquid inlet is not attached to a side wall. In some such embodiments, the liquid inlet is attached to a top wall.

Additionally or alternatively, in some such embodiments, the illumination assembly is not attached to the side wall. In some such embodiments, the illumination assembly is attached to a top wall, a bottom wall or both a top wall and a bottom wall.

Additionally or alternatively, in some such embodiments, the algae growth-substrate assembly is not attached to a side wall. In some such embodiments, the algae growth- substrate assembly is attached to a component selected from the group consisting of a top wall, a bottom wall, the illumination assembly and combinations thereof (e.g., any two or any three of the top wall, bottom wall and illumination assembly). Such embodiments are preferred as these allow assembly of the algae growth-substrate assembly separately, and subsequent addition of the side walls as a sleeve. Some such embodiments are preferred as these allow simple maintenance that involves removing the outer wall as a single unit, leaving the algae growth assembly (attached to one or more of the other components) accessible for maintenance.

In preferred embodiments, the algae growth-substrate assembly is attached to (and preferably supported by) the illumination assembly. In preferred such embodiments, the device is configured to allow removal of the side wall while the algae growth-substrate assembly remains attached to the illumination assembly. In some such embodiments, at least some of the algae growth-substrate contacts the inner walls of the vessel but is not attached thereto. For example, in embodiments where the illumination assembly comprises a central illumination unit (as described above) and/or multiple off-center elongated illumination units, the algae growth-substrate assembly is secured to the illumination units. For example, in some such embodiments, brackets are used to attach ribs (analogous to ribs known in the art if umbrellas) to the central unit and/or off-center elongated illumination units at various heights. Algae growth-substrates are then either secured to or laid on the ribs to keep the substrates suspended inside the inner volume at the desired height and position without attachment to the side walls. In some such embodiments, the illumination assembly is

### Algae Growth-Substrate

As noted above, a device according to the teachings herein comprises, inside the inner volume and below the liquid inlet, an algae growth-substrate assembly so that liquid released inside the inner volume through the liquid inlet falls onto the algae growth-substrate assembly.

In preferred embodiments, the algae growth-substrate assembly comprises a pair of algae growth-substrates:
an inwards-sloped substrate which directs liquid flowing on an upper surface thereof radially-inwards; and
an outwards-sloped substrate which directs liquid flowing on an upper surface thereof radially-outwards,
   wherein the outwards-sloped substrate is located below and separated from the inwards-sloped substrate so that liquid from the inwards-sloped substrate falls onto the upper surface of the outwards-sloped substrate.

In some embodiments, the horizontal dimensions of the inwards-sloped substrate is smaller than that of the outwards-sloped substrate (the outwards-sloped substrate is wider). Alternatively, the horizontal dimensions of the inwards-sloped substrate is larger than that of the outwards-sloped substrate (the inwards-sloped substrate is wider). In some embodiments, the horizontal dimensions of the inwards-sloped substrate is about the same (within 10%) than that of the outwards-sloped substrate.

In some embodiments, the substrate assembly comprises a single pair of inwards-sloped/outwards-sloped substrate. In such embodiments, liquid that flows downwards from the outwards-sloped substrate falls towards the bottom of the inner volume towards the drain.

In preferred embodiments, the substrate assembly comprises at least two, at least four, at least six and even at least eight such pairs of algae growth-substrate at different heights in the inner volume, where a given pair is positioned above and/or below at least one other pair so that an inwards-sloped substrate of a lower pair is located below and separated from an outwards-sloped substrate of a higher pair so that liquid from the outwards-sloped substrate of a higher pair falls onto the upper surface of an inwards-sloped substrate of a lower pair. As a result, liquid that travels downwards inside the inner volume successively moves radially-inwards on an inwards-sloped substrate and subsequently radially outwards on an outwards-sloped substrate.

The separation between any two adjacent substrates (in the same pair or in two different pairs) is any suitable separation. If the separation is too small, algae growth may bridge the separation and thereby block light from reaching portions of the substrate so that the efficiency of the device is reduced. At the same time, the larger the separation, the fewer substrates there are in per unit length of inner volume and the less algae per unit volume of inner volume can be grown. In some embodiments, the separation is not less than 5 cm and not more than 30 cm. In some such embodiments, the separation is not less than 6 cm, not less than 7 cm and even not less than 8 cm. In some such embodiments, the separation is not more than 25 cm and even not more than 20 cm. In preferred embodiments, the separation between any two adjacent substrates (in the same pair or in two different pairs) is such that the heights of the bottom of the upper substrate and of the top of the lower substrate are within 5 cm of each other, more preferably within 2 cm of each other and even more preferably within 1 cm of each other. If there is overlap (between the height of lowest part of the higher of the two substrates and the height of the highest part of the lower of the two substrates) then one of the two substrates obscures light from the illumination module from reaching a portion of the other of the two substrates. If there is a gap (between the height of lowest part of the higher of the two substrates and the height of the highest part of the lower of the two substrates) then some light from the illumination module may not illuminate any substrate.

In some embodiments, all of the inwards-sloped substrates of a substrate assembly are within about 10% of the same size. Some such embodiments are preferred for devices where the side walls of the vessel are parallel with the vertical axis of the inner volume.

In some embodiments, all of the outwards-sloped substrates of a substrate assembly are within about 10% of the same size. Some such embodiments are preferred for devices where the side walls of the vessel are parallel with the vertical axis of the inner volume. Specifically, in some embodiments, in horizontal cross section, an outwards-sloped substrate of a lower pair of substrates is about the same size (within about 10%) as an outwards-sloped substrate of a upper pair and in horizontal cross section, an inwards-sloped substrate of a lower pair of substrates is about the same size (within about 10%) as an inwards-sloped substrate of an upper pair.

In some embodiments, the inwards-sloped substrates of a substrate assembly are of different sizes with bigger inward-sloped substrates closer to the bottom of the inner volume. Some such embodiments are preferred for devices having an inner volume with a trapeze or conical vertical cross section. Specifically, in some embodiments, in horizontal cross section, an outwards-sloped substrate of a lower pair of substrates is larger than an outwards-sloped substrate of a upper pair and in horizontal cross section, an inwards-sloped substrate of a lower pair of substrates is larger than an inwards-sloped substrate of an upper pair.

In some preferred embodiments, the large dimension upper periphery of one, some and preferably all of the inwards-sloped substrates make contact with the inner surfaces of the wall of the vessel. Such embodiments allow liquid that is flowing down the walls of the vessel to be collected by the inwards-sloped substrate and directed radially-inwards.

In some preferred embodiments, the large dimension lower periphery of one, some and preferably all of the outwards-sloped substrates do not make contact with the inner surfaces of the wall of the vessel. Such embodiments avoid the pooling of liquid that is flowing down the walls of the vessel between the wall and the periphery of the outwards-sloped substrate. In some embodiments, the periphery is separated from the wall by at least 0.5 cm and even by at least about 1 cm but nor more than about 5 cm and even not more than about 3 cm.

The shape of the substrates in cross section perpendicular to the vertical axis of the inner volume is any suitable shape. In some embodiments, the shape follows that of the inner volume, for example, if the cross section of the inner volume perpendicular to the vertical cross section is square, the substrates are square and if the cross section of the inner volume perpendicular to the vertical cross section is round (e.g., when the side walls of the device area tube), the substrates are round. In some preferred embodiments, the substrates are round and preferably centered around the vertical axis.

In some embodiments, the outwards-sloped substrates of a substrate assembly are of different sizes with bigger outwards-sloped substrates closer to the bottom of the inner volume. Some such embodiments are preferred for devices having an inner volume with a trapeze or conical vertical cross section.

An inwards-sloped substrate directs liquid flowing on an upper surface thereof radially-inwards. The shape of an inwards-sloped substrate is any suitable shape, in cross section having larger horizontal dimensions at an upper portion and smaller horizontal dimensions at a lower portion, similar to a cone or bowl oriented with the wide opening upwards. The shape of the inwards-sloped substrate is any suitable shape, in some embodiments selected from the group consisting of conical, pyramidal, paraboloid and an ovoid or spherical section. The shape of the inwards-sloped substrate in vertical cross-section, is any suitable shape, in some embodiments selected from the group consisting of V-shaped, U-shaped, parabolic and an oval or a circle arc. In preferred embodiments, an inwards-slowed substrate comprises a drain hole, typically at least about 80 mm² (equivalent to a 5 mm radius circle) and even at least about 320 mm² (equivalent to a 10 mm radius circle). Such a drain hole prevents algae that drops from a higher substrate from blocking a lower substrate. Preferably, the drain hole is located at the lowest portion of the inwards-sloped substrate.

An outwards-sloped substrate directs liquid flowing on an upper surface thereof radially-outwards. The shape of an outwards-sloped substrate is any suitable shape, in cross section having smaller horizontal dimensions at an upper portion and larger horizontal dimensions at a lower portion, similar to a cone or bowl with the wide opening downwards. The shape of the outwards-sloped substrate is any suitable shape, in some embodiments selected from the group consisting of conical, pyramidal, paraboloid and an ovoid or spherical section. The shape of the outwards-sloped substrate in vertical cross-section, is any suitable shape, in some embodiments selected from the group consisting of V-shaped, U-shaped, parabolic and an oval or a circle arc.

In preferred embodiments, at least one, preferably most and most preferably all of the lowest portion of the inwards-sloped substrates are centered along the vertical axis of the inner volume so that liquid flowing on an upper surface of the inwards-sloped substrate is directed radially-inwards towards the central axis. Additionally, in preferred embodiments, at least one, preferably most and most preferably all of the apex (highest portion) of the outwards-sloped substrates are centered along the vertical axis of the inner volume so that liquid flowing on an upper surface of the outwards-sloped substrate is directed radially-outwards from the central axis.

The height of a given inwards-sloped substrate (dimension parallel to the vertical axis, from the higher peripheral edge (the edge close to the inner walls of the vessel) and the lower part (e.g., at the drain hole rim)) is any suitable height. The height is typically related to the width of that inwards-substrate to ensure that the steepness of the inwards-sloped substrate. As used herein, by steepness is meant the ratio of the vertical drop (the dimension parallel to the vertical axis from the higher peripheral edge to the lowest portion of the substrate. When in vertical cross section a side of inwards-sloped substrate is a straight line, steepness is the slope of the straight line. Low steepness allows more substrates to be packed into a given height of inner volume, but, in some embodiments, a greater proportion of light from the illumination assembly either does not illuminate the substrates or illuminates the substrate surfaces at a small (glancing) angle. High steepness allows each substrate to have a greater surface area allowing more algae to grow, in some embodiments ensures that more light from the illumination assembly illuminates a substrate surface and is more effective in letting excess algae disconnect from the substrate to fall down to the drain rather than clog the device.

In some embodiments the steepness is at least about 0.5 (0.5 cm vertical drop for every 1 cm horizontal dimension) and not more than about 3 (4 cm vertical drop for every 1 cm horizontal dimension). In some embodiments, the steepness is at least about 1 and even at least about 1.5. In some embodiments, the steepness is not more than about 2.5 and even not more than about 2.

During operation of the device, the algae that grows on the substrates absorbs carbon dioxide (and, in some embodiments, other gases) from gas introduced via the gas inlet and nutrients from liquid introduced via the liquid inlet by photosynthesis that is supported by light from the illumination assembly. During operation of the device, any suitable algae may be present in attached to the substrates including red algae species, brown algae species and/or green algae species. In some embodiments, a specific type of algae is seeded in the device so that the device contains primarily or exclusively the specific type of algae. In some such embodiments, the specific algae species may be selected as a species suitable for processing a specific type of wastewater, or absorbing a gas other than carbon dioxide, or because of the desire to harvest the selected species type. In some embodiments, the growth and development of a specific type of algae is encouraged by selection of the introduced gas, the introduced liquid and/or the wavelengths of light that the illumination assembly emits.

The algae growth-substrates are any substrate that allows the attachment and growth of sessile algae (algae that is anchored to the substrate), for example, benthic algae.

In some embodiments, at least one, e.g., one, some or all, of the substrates are impermeable to water (i.e., water cannot flow therethrough) so that the liquid introduced via the liquid inlet flows primarily and even exclusively along the upper surfaces of the impermeable substrates.

In embodiments, at least one, e.g. one, some or all, of the substrates are permeable to water (i.e., water can flow therethrough) so that the liquid introduced via the liquid inlet can potentially flow along the upper surfaces, the lower surfaces, the bulk and through the permeable substrates. It is currently believed that it is preferred that all of the substrates be permeable substrates as it is believed that permeable substrate allow the greatest effective contact of algae with gas and liquid in the inner volume.

In some embodiments, a permeable substrate is selected from the group consisting of a net and textile (including a fabric) Suitable specific permeable substrates include textiles such as geotextile (preferably 2-5 mm thick), jute fabric, shading nets used in the construction of agriculture net houses, camouflage netting. Suitable nets (including meshes, webs and other apertured components) preferably preferably have holes of not less than about 7 mm² (equivalent to round holes having a 1.5 mm radius) and not greater than about 50 mm² (equivalent to round holes having a 8 mm radius), for example 20 mm² (equivalent to round holes having a 2.5 mm radius). In some embodiments, the permeable substrate is made of an organic material, such as jute fabric, as these are often readily available. That said, organic materials may be less preferred for some implementations as these may relatively quickly be digested during operation of the device. Preferred materials from which a substrate is made include polymers such as polyethylene, polypropylene, polyester and polyethylene terephthalate (PET). It is currently believed that PET is preferred as being the most resistant to the conditions in the inner volume of the device during operation.

In some embodiments, the inwardly-sloped substrates are water permeable while at least a portion of the outwardly-sloped substrates are water impermeable. In preferred such embodiments, the inwardly-sloped substrates are water permeable while the central portions the apex) of the outwardly-sloped substrates are water impermeable. In some embodiments, the apex of an inwards-sloped substrate is water impermeable. In some embodiments, the apex of an inwards-sloped substrate is water permeable.

An advantage of the teachings herein is that a device can be scaled for any use by increasing the height and/or horizontal dimensions of the inner volume, in order to increase the amount of algae that is present in the inner volume. If for some reason there is an upper limit on the size desired for a single device (e.g., expense of construction or maintenance, the difficulty of emplacing or transporting a large device, a desire to mass-produce modestly sized devices), multiple devices can be used together. In some embodiments, the devices are used in parallel that is to say each individual device receives input liquid and input gas via the liquid inlet and gas inlet, respectively, from the same source and each device operates independently.

Alternatively, in some embodiments, at least two devices operate in series as a system.

For example, in some embodiments used for carbon sequestration the carbon dioxide content of effluent gas from a first device is greater than desired (e.g., greater than 450 ppm, or greater than 425 ppm, or greater than 410 ppm). By directing the effluent gas of a first device to the gas inlet of a second device, the effluent gas of the second device is reduced to acceptable levels. Thus, in some embodiments, at least two of the at least two devices are arranged in series so that a gas outlet of a first of the two devices is in fluid communication with a gas inlet of a second of the two devices so that gas released from an inner volume of the first device is processed in an inner volume of the second device.

For example, in some embodiments used for wastewater processing, effluent liquid from a first device includes an unacceptable level of contamination so requires further processing. By directing the effluent liquid of a first device to the liquid inlet of a second device, the effluent liquid of the second device is reduced to acceptable levels. Thus, in some embodiments, at least two of the at least two devices are arranged in series so that a drain of a first of the two devices is in fluid communication with a liquid inlet of a second of the two devices so that liquid released from an inner volume of the first device is processed in an inner volume of the second device.

Some embodiments of the teachings herein can be advantageously installed as air-scrubber devices including for carbon sequestration. Specifically compact and relatively light weight embodiments (such as embodiments that include a tube defining a side wall can easily be attached to a wall, including an external wall, of a chimney or smokestack of a carbon-dioxide releasing process (e.g., a powerplant, industrial process, chemical process) and the air inlet of the device functionally-associated with the flue thereof. The carbon-dioxide and other pollution in the flue gases are sequestered by the algae in the device.

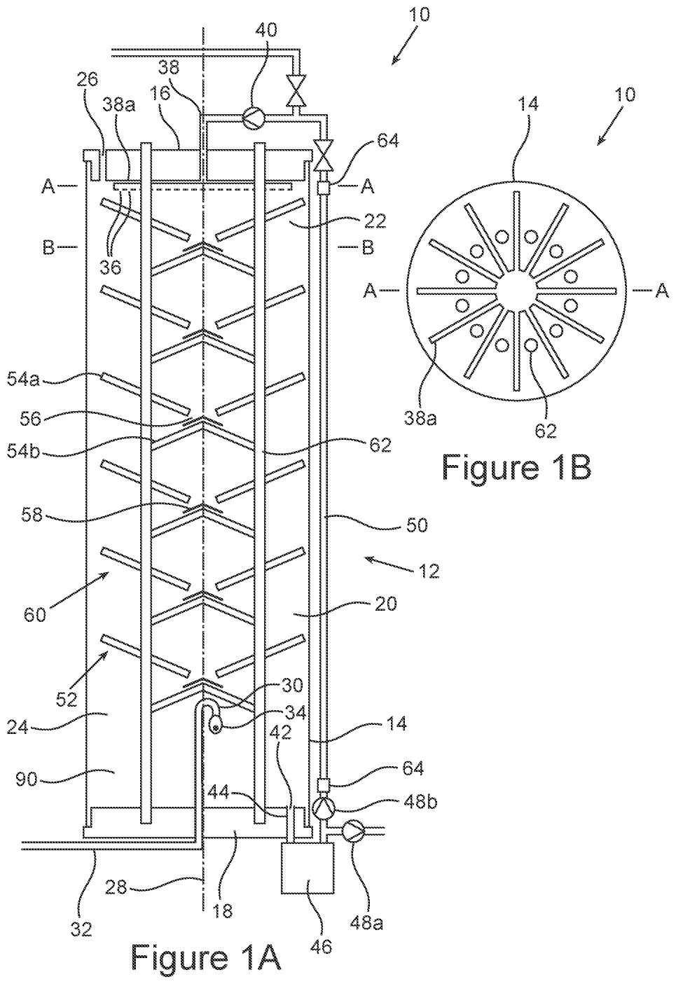

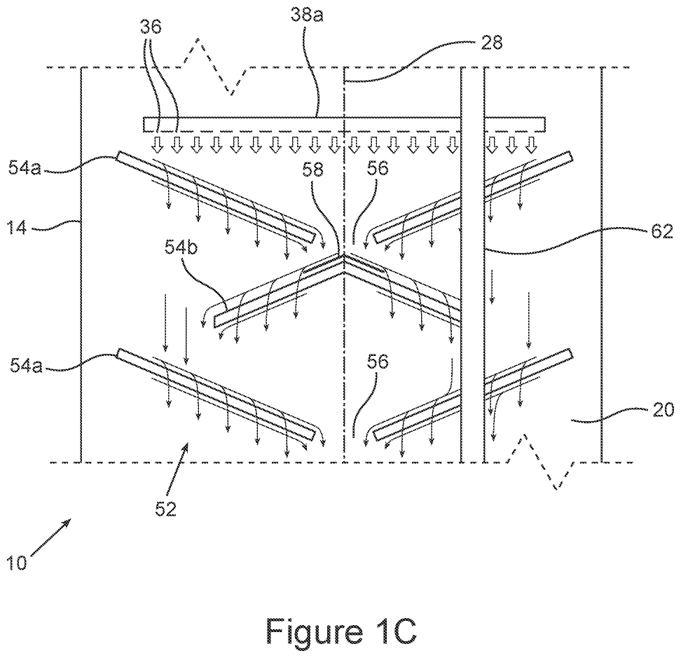

An exemplary embodiment of a device according to the teachings herein, device 10, is schematically depicted in FIGS. 1, in vertical cross section (FIG. 1A), horizontal cross section along A-A (FIG. 1B) and partial vertical cross section showing a presumed flow of liquid in the device during use (FIG. 1C). Device 10 comprises a vessel 12 having a tubular side wall 14 (an opaque 16″ (40.6 cm) diameter CPVC water pipe), a top wall 16 and a bottom wall 18 (both CPVC plus that snugly fit into pipe 14. Walls 14, 16 and 18 define an inner volume 20 having a top 22, a bottom 24, a height of 44″ (112 cm), a vertical axis 28, a rectangular vertical cross section that includes vertical axis 28 and a circular horizontal cross section perpendicular to vertical axis 28.

Device 10 includes a gas inlet 30 located inside inner volume 20, functionally-associated with a gas conduit 32 so that a gas that passes through gas conduit 32 is released inside inner volume 20 through gas inlet 30. Gas conduit 32 passes through bottom wall 18 Gas inlet 30 is provided with a float valve 34. If the water that accumulates at bottom of inner volume 20 reaches the level of gas inlet 30, float valve 34 prevents water from entering gas conduit 32 through gas inlet 30. Excess gas can exit inner volume 20 through gas outlet 26, a hole that that passes through top wall 16.

Device 10 further includes a plurality of liquid inlets 36 located inside inner volume 20, functionally-associated with a liquid conduit 38 so that a liquid that passes through liquid conduit 38 is released inside inner volume 20 through liquid inlets 36. In device 10, liquid conduit 38 includes a twelve-armed manifold 38a, each arm of manifold 38a including ten 2 mm holes each constituting a liquid inlet 36. Liquid that passes through liquid conduit 38 enters the arms of manifold 38a and exits through the liquid inlets 36 as 120 independent streams or sprays distributed across the entire horizontal cross section of top 22 of inner volume 20. functionally-associated with liquid conduit 38 is a pump 40 to drive liquid into liquid conduit 38.

Inside inner volume 20 is a drain 42 functionally-associated with a drain conduit 44 so that a liquid in inner volume 20 that enters drain 42 passes into drain conduit 44 to an algae harvester 46. Drain conduit 44 passes through bottom wall 18. Algae harvester 46 removes algae from the liquid and directs via a pump 48a to be discarded/further processed or via a pump 48b into a recycling conduit 50 to be recycled back into inner volume 20 via liquid conduit 38.

Inside inner volume 20 and below manifold 38a and liquid inlets 36 is an algae growth-substrate assembly 52. Liquid released inside inner volume 20 through liquid inlets 36 falls onto algae growth-substrate assembly 52. algae growth-substrate assembly 52 comprises six pairs of algae growth-substrates 54, each pair including:
an inwards-sloped substrate 54a which directs liquid flowing on an upper surface thereof radially-inwards (towards vertical axis 28); and
an outwards-sloped substrate 54b which directs liquid flowing on an upper surface thereof radially-outwards (away from vertical axis 28),
   wherein outwards-sloped substrate 54b is located below and separated from inwards-sloped substrate 54a so that liquid from inwards-sloped substrate 54a falls onto an upper surface of outwards-sloped substrate 54b.

Substrates 54a and 54b are made from sheets of 3 mm PET nets (available from DCL, Hebei, China) having 0.5 cm hexagonal holes. The sheets making up inwards-sloped substrates 54a are folded into an inverted conical shape with a 3-cm drain hole 56 centered around vertical axis 28. The sheets making up outwards-sloped substrates 54b are folded into a conical shape with the apex pointed upwards along vertical axis 28. Capping the apex of each outwards-sloped substrate 54b is an impermeable polyethylene cap 58.

Inside inner volume 20 is an illumination assembly 60 configured, when activated, to illuminate inner volume 20 from inside with sufficient light for algal photosynthesis. Illumination assembly 60 comprises twelve physically-separate illumination units 62, each illumination unit 62 comprising a transparent PMMA tube, 30 mm diameter with a 20 mm central bore. Inside each central bore along the entire length thereof are two strips of LEDs as light sources, each strip having 300 LED/meter, one strip producing red light and one strip producing blue light when activated. The bottom end of each illumination unit 62 snugly fits in a socket in bottom wall 18. The top end of each illumination unit 62 passes through top wall 16, allowing easy access to the LED strips for providing electricity and for maintenance without disassembly of other components of device 10. Illumination units 62 are oriented in parallel to vertical axis 28 and are located midway (50% of the distance) between vertical axis 28 and the inner surface of side wall 14.

Not depicted are brackets that encircle illumination units 62 and to which are connected ribs of PMMA profile that extend radially outwards and inwards. Substrates 54a and 54b rest on the PMMA ribs and are optionally secured thereto with plastic ties.

Device 10 is configured to allow simple separation of side wall 14. Specifically, connectors 64 on recycling conduit 50 are released, allowing recycling conduit 50 to be separated from device 10. Top wall 16 is then lifted upwards, together with liquid conduit 38 and manifold 38a. Side wall 14 is then lifted upwards, leaving illumination assembly 60 and substrate assembly 52 exposed for maintenance.

Device 10 is depicted in FIG. 1C is depicted in a schematic partial vertical cross section, where the arrows show a presumed flow of liquid in device 10 during use. Without being held to any one theory, it is currently believed that liquid inlets 36 introduce liquid into inner volume 20 as multiple streams or spray that fall onto the top surface of a inwards-sloped substrate 54a and some also on impermeable cap 58 at the apex of outwards-sloped substrate 54b. Some of the liquid falls radially inwards along the top surface of inwards-sloped substrate 54b until reaching drain hole 56 and then falls through the air, eventually hitting impermeable cap 58 at the apex of outwards-sloped substrate 54b. Some of the liquid that flows along the upper surface of inwards-sloped substrate 54a flows through the apertures in inwards-sloped substrate 54a and then either flows radially inwards along the bottom surface of inwards-sloped substrate 54a until reaching drain hole 56, or falls through the air to either land on the upper surface of another inwards-sloped substrate 54a or onto outwards-sloped substrate 54b. Liquid that falls on impermeable cap 58 at the apex of outwards-sloped substrate 54b flows radially-outwards to the permeable portions of outwards-sloped substrate 54b. Some liquid that falls on the permeable portions of outwards- sloped substrate 54b flows radially-outwards towards the periphery of outwards-sloped substrate 54b and then falls through the air, eventually landing on a lower inwards-sloped substrate 54a. Some of the liquid that flows along the upper surface of outwards-sloped substrate 54b flows through the apertures in outwards-sloped substrate 54b and then either flows radially outwards along the bottom surface of outwards-sloped substrate 54b until falling from the periphery thereof, or falls through the air to either land onto the upper surface of another inwards-sloped substrate 54a. Some liquid presumably also flows down along the surface of illumination unit 62. In such a way, liquid that passes through the algae growth-substrate assembly 52 is maintained as relatively thin film having a high-surface area with occasional mixing when falling from substrate to substrate, that allows the release of oxygen from the liquid and the absorption of carbon dioxide from gas in the inner volume. When flowing on, under or through a substrate, the liquid is in intimate contact with sessile algae growing on the substrate. Further, the surfaces of the substrates are illuminated with light from the illumination assembly with few, if any, shaded areas, the thin film of liquid allowing the light to penetrate to the algae with little absorption by the water.

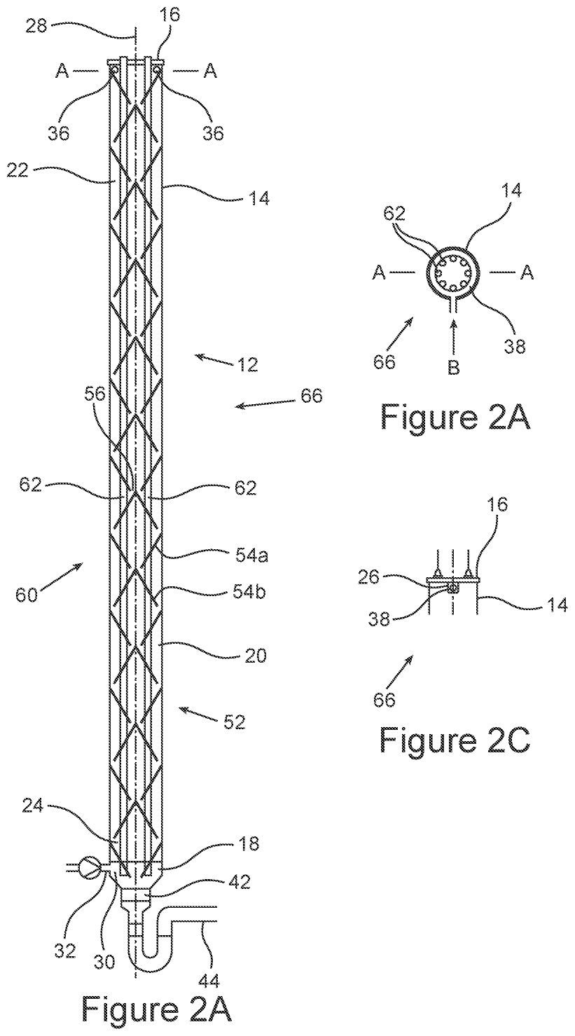

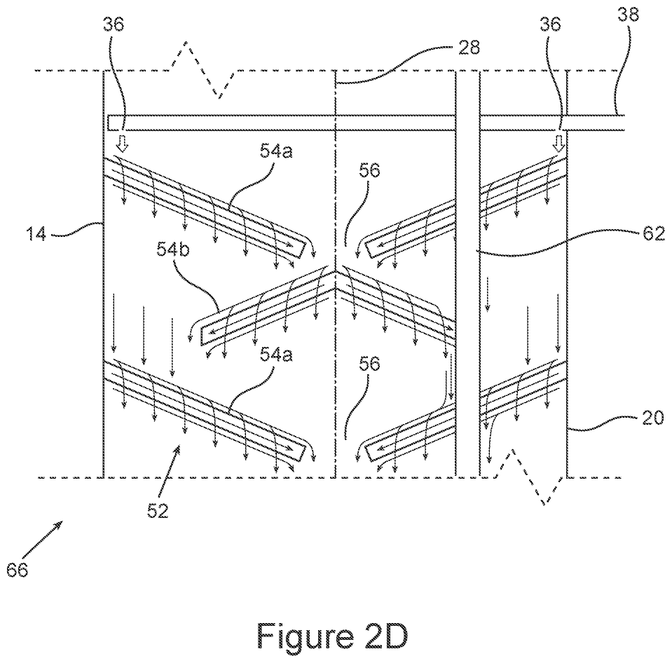

An additional exemplary embodiment of a device according to the teachings herein, device 66, is schematically depicted in FIGS. 2, in vertical cross section (FIG. 2A), horizontal cross section along A-A (FIG. 2B), partial side view from direction B (FIG. 2C) and partial vertical cross section showing a presumed flow of liquid in the device (FIG. 2D).

Device 66 is similar to device 10 discussed with reference to FIGS. 1 but includes a few noteworthy differences, some such differences mentioned hereinbelow.

Device 66 is particularly suitable for carbon sequestration and is a devoid of a recycling conduit.

Side wall 14 is a 300 cm long 20 cm diameter visible and UV light opaque CPVC water pipe.

Liquid that reaches the bottom 24 of inner volume 20 does not accumulate but immediately passes through drain 42 and to drain conduit 44.

Gas inlet 30 and gas conduit 32 are functionally-associated with a pump to drive gas to be processed through gas inlet 30 into inner volume 20.

Illumination assembly 60 includes eight physically separate illumination units 62 which top ends are flush with top wall 16. The upper ends of illumination units 62 are plugged with plugs that carry electrical leads to the LEDs that are inside illumination units 62.

Liquid conduit 38 is 25 mm OD 23 mm ID CPVC water pipe at the end of which is a complete loop which is located inside inner volume 20 at the very top, the top surface of the loop contacting the bottom of top wall 16. Liquid conduit 38 enters inner volume 20 through a notch in side wall 14 which alsoconstitutes air outlet 38 of device 36. On the bottom side of the loop are a plurality of 2 mm holes that constitute water inlets 36.

Algae growth-substrate assembly 52 includes ten pairs of inwards-sloped substrates 54a/outwards-sloped substrates 54b, while the lowest substrate is an additional non-paired inwards-sloped substrate 54a. All substrates are made of commercially-available 1 mm thick shade house mesh made of knitted polyethylene.

The upper periphery of inwards-sloped substrates 54a contacts the inner face of side wall 14. In horizontal cross section, each inwards-sloped substrate 54a has a ring-shaped cross section with a radius of 10 cm having a 2.5 cm radius drain hole 56. The height of each inwards-sloped substrate 54a is 13 cm from the top of the periphery where the substrate touches the inner face of side wall 14 to the rim of drain hole 56. As a result, the steepness of the inwards-sloped substrates 54a is 13/7.5=1.73. Since in vertical cross section inwards-sloped substrates 54a are a straight line, the steepness is also the slope.

Outwards-sloped substrates 54b are entirely permeable and are devoid of an impermeable cap 58. The lower periphery of outwards-sloped substrates 54b is 1 cm from the inner surface of side wall 14. In horizontal cross section, each outwards-sloped substrate 54b has a circular cross section with a radius of 9 cm. The height of each outwards-sloped substrate 54b is 15 cm from the apex to the bottom of the periphery. As a result, the steepness of the outwards-sloped substrates 54b is 15/9=1.66. Since in vertical cross section outwards-sloped substrates 54b are a straight line, the steepness is also the slope.

Device 66 is depicted in FIG. 2D in a schematic partial vertical cross section, where the arrows show a presumed flow of liquid in device 66 during use. Without being held to any one theory, it is currently believed that the flow is substantially similar to the discussed above for device 10 with reference to FIG. 1C. A first substantial difference is that liquid enters the internal volume at multiple locations, but all locations are close to the inner surface of side wall 14. A second substantial difference is that the outwards-sloped substrate 54b is entirely permeable, being devoid of an impermeable cap 58.

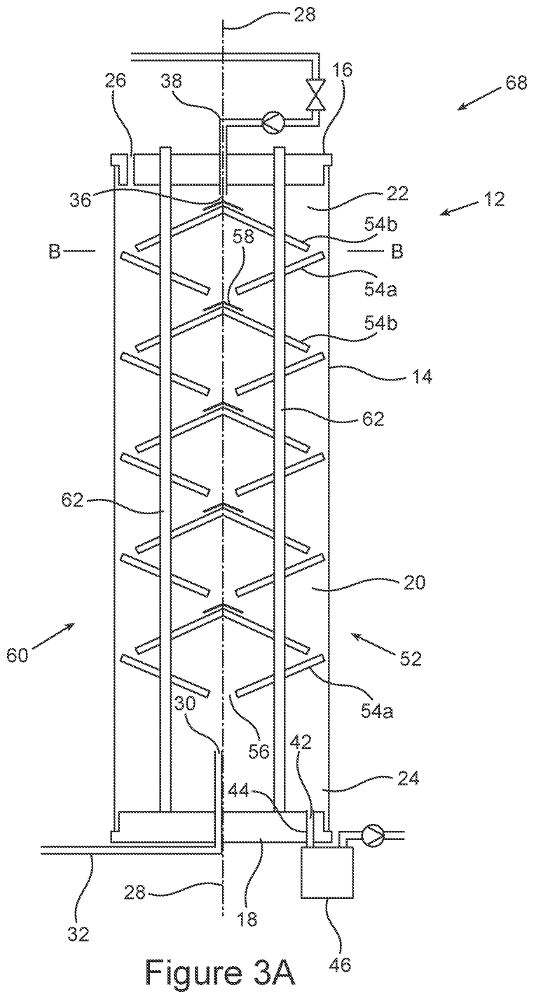

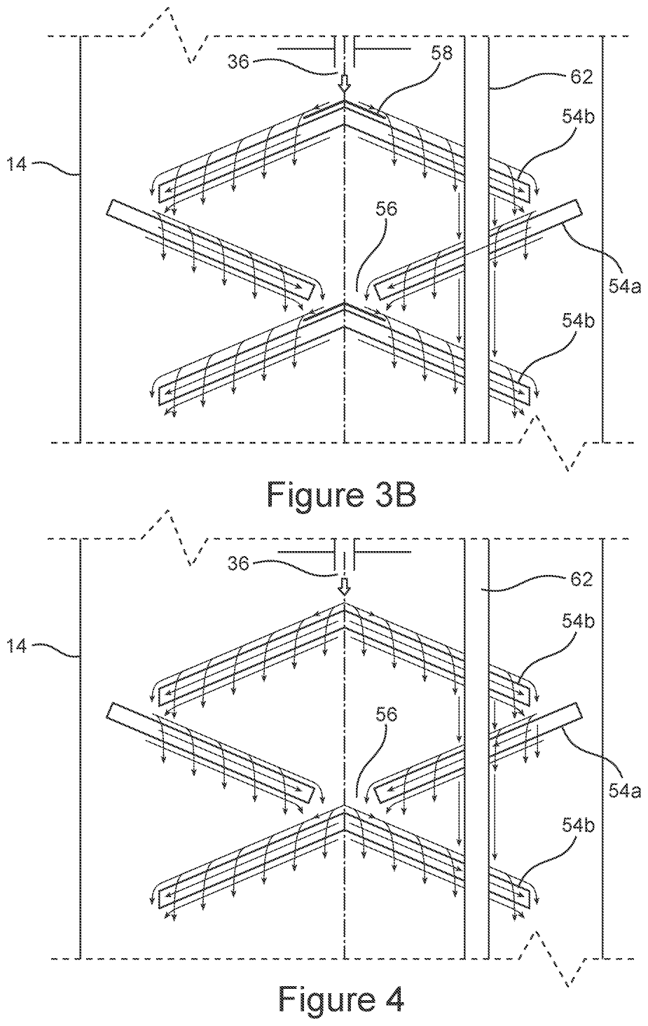

An additional exemplary embodiment of a device according to the teachings herein, device 68, is schematically depicted in FIGS. 3, in vertical cross section (FIG. 3A) and schematic partial vertical cross section showing a presumed flow of liquid in the device (FIG. 3B).

Device 68 is similar to device 10 discussed with reference to FIGS. 1 but includes a few noteworthy differences, some such differences mentioned hereinbelow.

Algae growth-substrate assembly 52 of device 68 includes four pairs of inwards-sloped substrates 54a/outwards-sloped substrates 54b. The lowest substrate is an additional non-paired inwards-sloped substrate 54a. The uppermost substrate is an additional non-paired outwards-sloped substrate 54b. All substrates 54a/54b are made of commercially-available 4 mm thick polyester geotextile.

Device 68 includes a single liquid inlet 36. Liquid introduced into inner volume 20 of device 68 through liquid inlet 36 falls on impermeable cap 58 at the apex of the uppermost non-paired outwards-sloped substrate 54b and flows radially-outwards to the permeable portions of outwards-sloped substrate 54b.

Device 68 is depicted in FIG. 3B in a schematic partial vertical cross section, where the arrows show a presumed flow of liquid in device 68 during use. Without being held to any one theory, it is currently believed that the flow is substantially similar to the discussed above for device 10 with reference to FIG. 1C. A first substantial difference is that liquid enters the internal volume at a single location from a single liquid inlet 36, but is distributed radially outwards by the uppermost unpaired outwards-sloped substrate 54b. A second substantial difference is that, because substrates 54a and 54b are made of geotextile, it is believed that some liquid flows inside the bulk of the substrates.

In FIG. 4 is depicted in a schematic partial vertical cross section a device 70, a variant of device 68 discussed with reference to FIGS. 3. The only substantial difference is that the outwards-sloped substrates 54b are entirely permeable, being devoid of an impermeable cap 58, similar to the described for device 66 with reference to FIGS. 2.

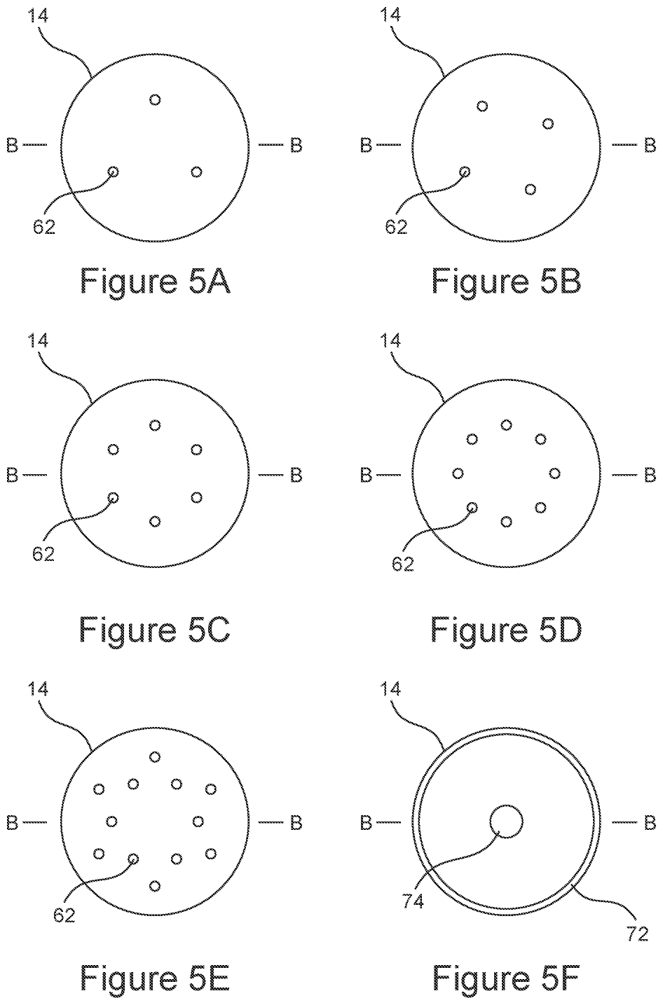

FIGS. 5A to 5F each depict an embodiment of a device as described herein in horizontal cross section, each showing an embodiment with a different illumination assembly.

In FIG. 5A, a device having three physically-separated illumination units 62, each illumination unit 62 comprising an elongated transparent container having a longitudinal axis and a horizontal cross section, each container containing a light source that, when activated emits light that passes through walls of the transparent container. Illumination units 62 are as described above with reference to devices 10 and 66.

In FIG. 5B, a device having four physically separated elongated illumination units 62 is schematically depicted.

In FIG. 5C, a device having six physically separated elongated illumination units 62 is schematically depicted.

In FIG. 5D, a device having eight physically separated elongated illumination units 62 is schematically depicted.

In FIG. 5E, a device having twelve physically separated elongated illumination units 62 is schematically depicted.

In FIG. 5F, a device having a peripheral illumination unit 72 and a central illumination unit 74.

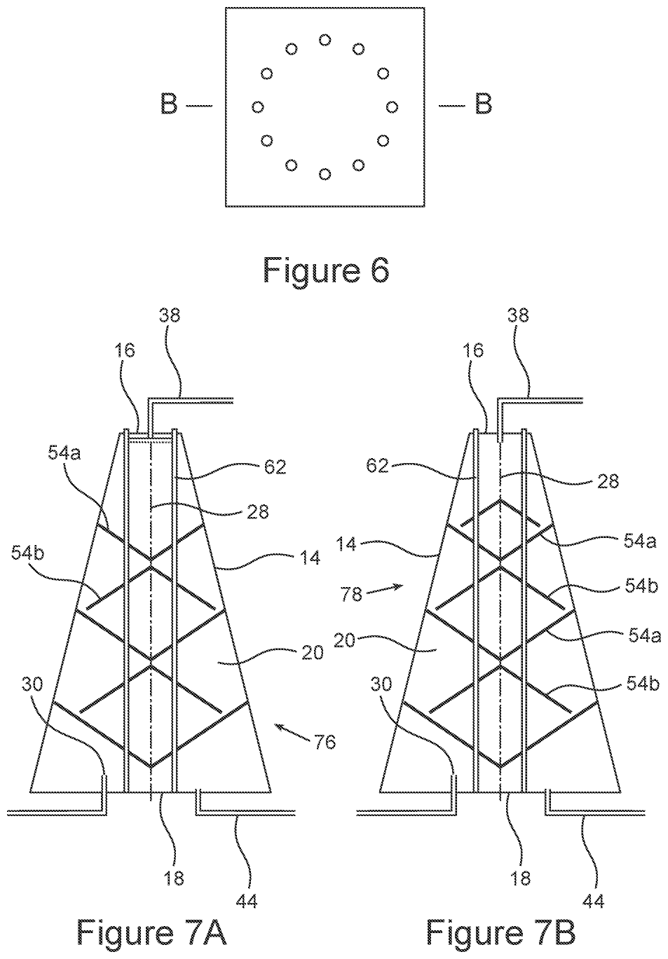

The devices previously described hereinabove with reference to FIGS. 1 to 5 all have circular horizontal cross sections. In FIG. 6 is schematically depicted a device having a square horizontal cross section and twelve physically separated elongated illumination units 62.

The devices previously described hereinabove with reference to FIGS. 1 to 5 all have a tubular side wall 14 with a rectangular vertical cross section. For each device, the dimensions of the inwards-sloped substrates are substantially the same and the dimensions of the outwards-sloped substrates are substantially the same. In FIG. 7A is depicted a device 76 and in FIG. 7B is depicted a device 78. Devices 76 and 78 both have a truncated-conical side wall having a circular horizontal cross section and a trapeze vertical cross section. In both devices, the lower inwards-sloped substrates 58a are larger than the higher inwards-sloped substrates 58a and the lower outwards-sloped substrates 58b are larger than the higher outwards-sloped substrates 58b.

Unless otherwise defined, all technical and scientific terms used herein have the same meaning as commonly understood by one of ordinary skill in the art to which the invention pertains. In case of conflict, the specification, including definitions, takes precedence.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”. As used herein, the indefinite articles “a” and “an” mean “at least one” or “one or more” unless the context clearly dictates otherwise.

As used herein, when a numerical value is preceded by the term “about”, the term “about” is intended to indicate +/−10%. As used herein, a phrase in the form “A and/or B” means a selection from the group consisting of (A), (B) or (A and B). As used herein, a phrase in the form “at least one of A, B and C” means a selection from the group consisting of (A), (B), (C), (A and B), (A and C), (B and C) or (A and B and C).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

Section headings are used herein to ease understanding of the specification and should not be construed as necessarily limiting.

1.-29. (canceled)

30. A device suitable for growing algae comprising:
a. a vessel having walls defining an inner volume, said inner volume having a top, a bottom, a height, a vertical axis, a vertical cross section including said vertical axis and a horizontal cross section perpendicular to said vertical axis;
b. a gas inlet located inside said inner volume functionally-associated with a gas conduit, so that a gas that passes through said gas conduit is released inside said inner volume through said gas inlet;
c. a liquid inlet located inside said inner volume functionally-associated with a liquid conduit, so that a liquid that passes through said liquid conduit is released inside said inner volume through said liquid inlet;
d. inside said inner volume and below said liquid inlet, an algae growth-substrate assembly so that liquid released inside said inner volume through said liquid inlet falls onto said algae growth-substrate assembly;
e. inside said inner volume and below said algae growth-substrate, a drain functionally-associated with a drain conduit so that a liquid in said inner volume that enters said drain passes into said drain conduit; and
e. inside said inner volume, an illumination assembly configured, when activated, to illuminate said inner volume from inside said inner volume with sufficient light for algal photosynthesis,
wherein said algae growth-substrate assembly comprises a pair of algae growth-substrates:
an inwards-sloped substrate which directs liquid flowing on an upper surface thereof radially-inwards; and
an outwards-sloped substrate which directs liquid flowing on an upper surface thereof radially-outwards,
wherein said outwards-sloped substrate is located below and separated from said inwards-sloped substrate so that liquid from said inwards-sloped substrate falls onto said upper surface of said outwards-sloped substrate.

31. The device of claim 30, wherein said illumination assembly comprises at least three physically-separated illumination units, each said illumination unit comprising an elongated transparent container having a longitudinal axis and a horizontal cross section, each said container containing a light source that, when activated, emits light that passes through walls of said transparent container to illuminate said inner volume, wherein each said illumination unit is located inside said inner volume with said longitudinal axis oriented within 10° of parallel with said vertical axis of said inner volume, wherein each said illumination unit passes through a portion of said substrate assembly, does not intersect with said vertical axis of said inner volume and is devoid of contact with said walls of said vessel.

32. The device of claim 30, said illumination assembly comprising at least one illumination unit, each said illumination unit including a transparent container located inside said inner volume, said transparent container containing a light source that, when activated, emits light that passes through walls of said transparent container.to illuminate said inner volume from inside said inner volume.

33. The device of claim 32, wherein in at least one said illumination unit, a light source is integrally formed with a respective transparent container.

34. The device of claim 32, wherein in at least one said illumination unit, a light source is separable from a respective transparent container.

35. The device of claim 30, said algae growth-substrate assembly comprising at least two said pairs of algae growth-substrates at different heights in said inner volume, where a given said pair is positioned above and/or below at least one other said pair so that an inwards-sloped substrate of a lower pair is located below and separated from an outwards-sloped substrate of a higher pair so that liquid from said outwards-sloped substrate of said higher pair falls onto an upper surface of said inwards-sloped substrate of a lower pair.

36. The device of claim 30, wherein at least one said substrate is impermeable to water.

37. The device of claim 30, wherein at least one said substrate is permeable to water.

38. The device of claim 37, wherein said permeable substrate is selected from the group consisting of a net and textile.

39. A waste processing system comprising at least two devices of claim 30, wherein at least one of:
at least two of said at least two devices are arranged in series so that a gas outlet of a first of said two devices is in fluid communication with a gas inlet of a second of said two devices so that gas released from an inner volume of said first device is processed in an inner volume of said second device; and
at least two of said at least two devices are arranged in series so that a drain of a first of said two devices is in fluid communication with a liquid inlet of a second of said two devices so that liquid exiting the inner volume of said first device is processed in an inner volume of said second device.

40. A device suitable for growing algae comprising:
a. a vessel having walls defining an inner volume, said inner volume having a top, a bottom, a height, a vertical axis, a vertical cross section including said vertical axis and a horizontal cross section perpendicular to said vertical axis;
b. a gas inlet located inside said inner volume functionally-associated with a gas conduit so that a gas that passes through said gas conduit is released inside said inner volume through said gas inlet;
c. a liquid inlet located inside said inner volume functionally-associated with a liquid conduit so that a liquid that passes through said liquid conduit is released inside said inner volume through said liquid inlet;
d. inside said inner volume and below said liquid inlet, an algae growth-substrate assembly so that liquid released inside said inner volume through said liquid inlet falls onto said algae growth-substrate assembly;
e. inside said inner volume and below said algae growth-substrate, a drain functionally-associated with a drain conduit so that a liquid in said inner volume that enters said drain passes into said drain conduit; and
e. inside said inner volume, an illumination assembly configured, when activated, to illuminate said inner volume from inside said inner volume with sufficient light for algal photosynthesis,
wherein said illumination assembly comprises at least three physically-separated illumination units, each said illumination unit comprising an elongated transparent container having a longitudinal axis and a horizontal cross section, each said container containing a light source that, when activated, emits light that passes through walls of a said transparent container to illuminate said inner volume, wherein each said illumination unit is located inside said inner volume with said longitudinal axis oriented within 10° of parallel with said vertical axis of said inner volume, wherein each said illumination unit passes through a portion of said substrate assembly, does not intersect with said vertical axis of said inner volume and is devoid of contact with said walls of said vessel.

41. The device of claim 40, wherein said algae growth-substrate assembly comprises a pair of algae growth-substrates:
an inwards-sloped substrate which directs liquid flowing on an upper surface thereof radially-inwards; and
an outwards-sloped substrate which directs liquid flowing on an upper surface thereof radially-outwards,
wherein said outwards-sloped substrate is located below and separated from said inwards-sloped substrate so that liquid from said inwards-sloped substrate falls onto said upper surface of said outwards-sloped substrate.

42. The device of claim 40, wherein a distance of a said longitudinal axis of at least one of said illumination units of said illumination assembly from said vertical axis of said inner volume is not less than about 20% and note more than about 80% of the distance from said vertical axis of said inner volume to an inner surface of said walls of said vessel.

43. The device of claim 40, said inner volume being cylindrical.

44. The device of claim 40, said walls of said vessel comprising a side wall.

45. The device of claim 44, said side wall comprising a tube.

46. The device of claim 44, wherein said illumination assembly is not attached to said side wall.

47. The device of claim 44, wherein said algae growth-substrate assembly is not attached to said side wall.

48. The device of claim 47, wherein said algae growth-substrate assembly is attached to said illumination assembly.

49. The device of claim 48, wherein the device is configured to allow removal of said side wall while said algae growth-substrate assembly remains attached to said illumination assembly.
